(19) United States
(12) Patent Application Publication
(10) Pub. No.: US 2008/0280862 A1
(43) Pub. Date: Nov. 13, 2008

(54) Methods, compositions, and kits for the treatment of pain

(76) Inventors: Jennifer M. Cermak, North Reading, MA (US); Maura T. Brescia, Somerville, MA (US); Christine Bunt, Lexington, MA (US); M. James Nichols, Boston, MA (US)

Correspondence Address: CLARK & ELBING LLP, 101 FEDERAL STREET, BOSTON, MA, 02110, US

(21) Appl. No.: 12/006,648

(22) Filed: Jan. 4, 2008

Related U.S. Application Data
(60) Provisional application No. 60/878,905, filed on Jan. 5, 2007.
(60) Provisional application No. 60/880,556, filed on Jan. 16, 2007.

(51) Int. Cl. A61K 31/56; A61P 19/04; A61P 29/00

(52) U.S. Cl. 514/171

(57) ABSTRACT

The invention features methods, compositions, and kits for the treatment of pain.

### CROSS-REFERENCE TO RELATED APPLICATIONS

This application claims benefit of U.S. Ser. No. 60/878,905, filed Jan. 5, 2007, and U.S. Ser. No. 60/880,556, filed Jan. 16, 2007, each of which is hereby incorporated by reference.

### FIELD OF THE INVENTION

This invention relates to the treatment of pain.

### BACKGROUND OF THE INVENTION

The sensation of pain is a common symptom that may be indicative of an underlying disease or injury, or the expression of an abnormal function within the nervous system. Pain is often the primary incentive for which treatment is sought.

Pain can take a variety of forms depending on its origin. Pain may be described as being peripheral neuropathic if the initiating injury occurs as a result of a complete or partial transection or lesion to of a nerve or trauma to a nerve plexus. Alternatively, pain is described as being central neuropathic following a lesion to the central nervous system, such as a spinal cord injury or a cerebrovascular accident. Inflammatory pain is a form of pain that is caused by tissue injury or inflammation (e.g., in postoperative pain or rheumatoid arthritis). Following a peripheral nerve injury, symptoms are often experienced in a chronic fashion, distal to the site of injury and are characterized by hyperesthesia (enhanced sensitivity to a natural stimulus), hyperalgesia (abnormal sensitivity to a noxious stimulus), allodynia (widespread tenderness, associated with hypersensitivity to normally innocuous tactile stimuli), and/or spontaneous burning or shooting lancinating pain. In inflammatory pain, symptoms are apparent, at least initially, at the site of injury or inflamed tissues and typically accompany arthritis-associated pain, musculo-skeletal pain, and postoperative pain. Nociceptive pain is the pain experienced in response to a noxious stimulus, such as a needle prick or during trauma or surgery. Functional pain refers to conditions in which there is no obvious peripheral pathology or lesion to the nervous system. This particular form of pain is generated by abnormal function of the nervous system and conditions characterized by such pain include fibromyalgia, tension-type headache, and irritable bowel syndrome. The different types of pain may coexist or pain may be transformed from inflammatory to neuropathic during the natural course of the disease, as in post-herpetic neuralgia.

### SUMMARY OF THE INVENTION

In one aspect, the invention features a method of treating pain in a subject including orally administering to the subject a corticosteroid and a tricyclic compound. In this method, the corticosteroid and tricyclic compound are orally administered simultaneously, or within 14 days of each other, in amounts that together are sufficient to treat the subject.

In another aspect, the invention features a kit including a tricyclic compound and instructions for orally administering the tricyclic compound and a corticosteroid simultaneously or within 14 days of each other to a subject having pain.

In another aspect, the invention features a kit including a corticosteroid and instructions for orally administering the corticosteroid and a tricyclic compound simultaneously or within 14 days of each other to a subject having pain.

In yet another aspect, the invention features a kit including a corticosteroid and a tricyclic compound and instructions for orally administering the corticosteroid and tricyclic compound simultaneously or within 14 days of each other to a subject having pain.

In another aspect, the invention features a composition including a corticosteroid and a tricyclic compound. In this aspect, the corticosteroid (e.g., prednisolone) or tricyclic compound (e.g., nortripytiline) can be formulated for delayed released (e.g., released at least 0.5, 1, 2, 3, 4, 5, 6, 7, 8, 9, 10, 11, or 12 hours after administration), controlled release, and/or immediate release. In this aspect either or both the corticosteroid and/or tricyclic compound can be formulated for both immediate and delayed release.

In any of the forgoing aspects, examples of pain are clinical pain, namely inflammatory pain, functional pain, nociceptive pain, and neuropathic pain (e.g., peripheral neuropathic pain), whether acute or chronic. Other exemplary conditions that may be treated in the forgoing aspects of the invention are pain associated with soft tissue, joint, bone inflammation and/or damage (e.g., acute trauma, osteoarthritis, or rheumatoid arthritis), myofascial pain syndromes (fibromylagia), headaches (including cluster headache, migraine and tension type headache), stump pain, myocardial infarction, angina, ischemic cardiovascular disease, post-stroke pain, sickle cell anemia, peripheral vascular occlusive disease, cancer, inflammatory conditions of the skin or joints, diabetic neuropathy, acute tissue damage from surgery or traumatic injury (e.g., burns, lacerations, or fractures), musculo-skeletal pain (after trauma, infections, and exercise), neuropathic pain caused by spinal cord injury, tumors, compression, inflammation, dental pain, episiotomy pain, deep and visceral pain (e.g., heart pain, bladder pain, or pelvic organ pain), muscle pain, eye pain, orofacial pain (e.g., odontalgia, trigeminal neuralgia, glossopharyngeal neuralgia), abdominal pain, gynecological pain (e.g., dysmenorrhea and labor pain), pain associated with nerve and root damage due to trauma, compression, inflammation, toxic chemicals, metabolic disorders, hereditary conditions, infections, vasculitis and autoimmune diseases, central nervous system pain, such as pain due to spinal cord or brain stem damage, cerebrovascular accidents, tumors, infections, demyelinating diseases including multiple sclerosis, spinal pain, chronic lower back pain (e.g., ankylosing spondylitis, degenerative disk disease, radiculapathy, and radicular pain), sciatica, chronic neck pain, post-operative pain (e.g., mastectomy and phantom limb pain), and pain associated with post-herpetic neuralgia, cancer, cystic fibrosis, HIV, and polymyalgia rheumatica.

In another aspect the compositions, kits, and methods of the invention include a tricyclic antidepressant and one or more of the following: anti-convulsants, muscle relaxants, pregabalin, gabapentin, ketamide, analgesics (e.g., opiods, NSAIDs, COX-2 inhibitors), other antidepressants (e.g., selective serotonin reuptake inhibitor (SSRIs)), cannibinoids, sedatives, and anti-anxiety drugs.

In another aspect the compositions, kits, and methods of the invention include a corticosteroid and one or more of the following: anti-convulsants, muscle relaxants, pregabalin, gabapentin, ketamide, analgesics (e.g., opiods, NSAIDs, COX-2 inhibitors), other antidepressants (e.g., SSRIs), cannibinoids, sedatives, and anti-anxiety drugs.

In another aspect the compositions, kits, and methods of the invention include a tricyclic antidepressant, a corticosteroid, and one or more of the following: anti-convulsants, muscle relaxants, pregabalin, gabapentin, ketamide, analgesics (e.g., opiods, NSAIDs, COX-2 inhibitors), other antidepressants (e.g., SSRIs), cannibinoids, sedatives, and anti-anxiety drugs.

Also in any of the forgoing aspects, the corticosteroids can include prednisone or prednisolone, and the tricyclic compounds can include nortriptyline, imipramine, or desipramine.

In certain embodiments of the compositions, kits, and methods of the invention, the only pharmacologically active agents in the composition or kit, or used in the method, are those recited. In this embodiment, pharmacologically inactive excipients may also be present in the composition.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “prednisolone” is meant the free base as well as any pharmaceutically acceptable salt thereof (e.g., prednisolone acetate).

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

By “corticosteroid” is meant any naturally occurring or synthetic compound characterized by a hydrogenated cyclopentanoperhydro-phenanthrene ring system and having immunosuppressive and/or anti-inflammatory activity. Naturally occurring corticosteroids are generally produced by the adrenal cortex. Synthetic corticosteroids may be halogenated. Examples corticosteroids are provided herein.

By “tricyclic compound” is meant a compound having one the formulas (I), (II), (III), or (IV):

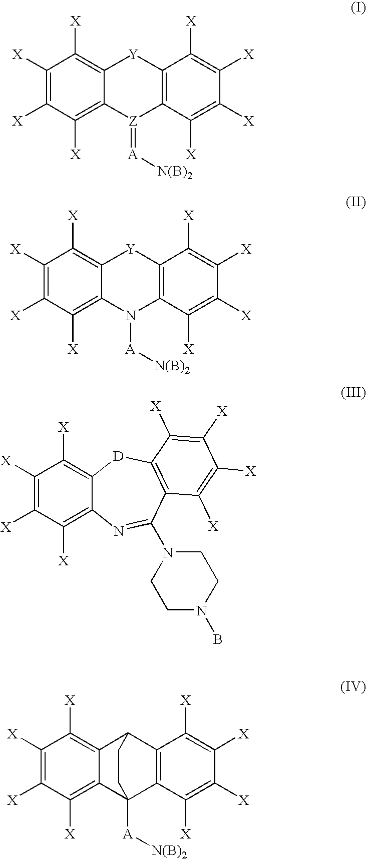

wherein each X is, independently, H, Cl, F, Br, I, CH₃, CF₃, OH, OCH₃, CH₂CH₃, or OCH₂CH₃; Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, or S(O)₀₋₂. In preferred embodiments, each X is, independently, H, Cl, or F; Y is (CH₂)₂, Z is C; A is (CH₂)₃; and each B is, independently, H, Cl, or F. Other tricyclic compounds are described below. Tricyclic compounds include tricyclic antidepressants such as amoxapine, 8-hydroxyamoxapine, 7-hydroxyamoxapine, loxapine (e.g., loxapine succinate, loxapine hydrochloride), 8-hydroxyloxapine, amitriptyline, clomipramine, doxepin, imipramine, trimipramine, desipramine, nortriptyline, and protriptyline, although compounds need not have antidepressant activities to be considered tricyclic compounds of the invention.

By “treating” is meant administering or prescribing a composition for the treatment or prevention of pain.

By “patient” or “subject” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “an amount sufficient” is meant the amount of a compound, in a combination of the invention, required to treat or prevent pain in a clinically relevant manner. A sufficient amount of an active compound used to practice the present invention for therapeutic treatment of pain varies depending upon the manner of administration, the age, body weight, and general health of the patient. Ultimately, the prescribers will decide the appropriate amount and dosage regimen. Additionally, an effective amount may can be that amount of compound in the combination of the invention that is safe and efficacious in the treatment of a patient having pain over each agent alone as determined and approved by a regulary authority (such as the U.S. Food and Drug Administration).

By “systemic administration” is meant all nondermal routes of administration, and specifically excludes topical and transdermal routes of administration.

By “controlled release” is meant that the therapeutically active component is released from the formulation over a defined period of time, such that at a given dose, the Cₘₐₓ is decreased. In controlled release formulations the Tₘₐₓ may or may not change.

By “delayed release” is meant that a substantial portion of the therapeutically active component is released from the formulation at least 2 hours after administration.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Other features and advantages of the invention will be apparent from the following detailed description, and from the claims.

### DETAILED DESCRIPTION

We have discovered that certain corticosteroids, when administered with certain tricyclic compounds, synergistically reduce pain in animal models of pain.

### Indications

The methods, compositions, and kits of the invention are useful for treating pain, including clinical pain, namely inflammatory pain, functional pain, nociceptive pain, and neuropathic pain (e.g., peripheral neuropathic pain), whether acute or chronic (e.g., pain lasting for greater than one, two, three, four, or more months). Conditions that may be associated with pain include, for example, soft tissue, joint, bone inflammation and/or damage (e.g., acute trauma, osteoarthritis, or rheumatoid arthritis), myofascial pain syndromes (fibromylagia), headaches (including cluster headache, migraine and tension type headache), stump pain, myocardial infarction, angina, ischemic cardiovascular disease, post-stroke pain, sickle cell anemia, peripheral vascular occlusive disease, cancer, inflammatory conditions of the skin or joints, diabetic neuropathy, and acute tissue damage from surgery or traumatic injury (e.g., burns, lacerations, or fractures). The present invention is also useful for the treatment, reduction, or prevention of musculo-skeletal pain (after trauma, infections, and exercise), neuropathic pain caused by spinal cord injury, tumors, compression, inflammation, dental pain, episiotomy pain, deep and visceral pain (e.g., heart pain, bladder pain, or pelvic organ pain), muscle pain, eye pain, orofacial pain (e.g., odontalgia, trigeminal neuralgia, glossopharyngeal neuralgia), abdominal pain, gynecological pain (e.g., dysmenorrhea and labor pain), pain associated with nerve and root damage due to trauma, compression, inflammation, toxic chemicals, metabolic disorders, hereditary conditions, infections, vasculitis and autoimmune diseases, central nervous system pain, such as pain due to spinal cord or brain stem damage, cerebrovascular accidents, tumors, infections, demyelinating diseases including multiple sclerosis, chronic lower back pain (e.g., ankylosing spondylitis, degenerative disk disease, radiculopathy, and radicular pain), sciatica, chronic neck pain, and post-operative pain (e.g., mastectomy, orthopedic and phantom limb pain). The present invention is also useful for treating pain associated with post-herpetic neuralgia, cancer, cystic fibrosis, HIV, and polymyalgia rheumatica.

### Tricyclic Compounds

Tricyclic compounds that can be used in the methods, compositions, and kits of the invention include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

Standard recommended dosages for several tricyclic antidepressants are provided in Table 1, below. Other standard dosages are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment of the invention, the tricyclic antidepressant may be administered a dose lower than the standard recommended dose. For example, nortriptyline can be administered at between 10-75 mg/day, 20-60 mg/day, or 30-50 mg/day.

TABLE 1

| Compound | Standard Dose |
|---|---|
| Nortriptyline | 75-150 mg/day |
| Desipramine | 100-200 mg/day |
| Imipramine | 10-200 mg/day |

### Corticosteroids

Corticosteroids that can be employed in a method, composition, or kit of the invention include corticosteroids include those from the class of selective glucocorticosteroid receptor agonists (SEGRAs), 11-alpha, 17-alpha,21-trihydroxypregn-4-ene-3,20-dione; 11-beta, 16-alpha, 17,21-tetrahydroxypregn-4-ene-3,20-dione; 11-beta,16-alpha,17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

### Steroid Receptor Modulators

Steroid receptor modulators (e.g., antagonists and agonists) may be used as a substitute for or in addition to a corticosteroid in the methods, compositions, and kits of the invention. Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

### Other Compounds

Other compounds that may be used as a substitute for or in addition to a corticosteroid in the methods, compositions, and kits of the invention A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

### Formulation of Pharmaceutical Compositions

The compositions, methods, and kits of the invention can include formulation(s) of compound(s) that, upon administration to a subject, result in a concentration of the compound(s) that reduces pain. The compound(s) may be contained in any appropriate amount in any suitable carrier substance, and are generally present in an amount of 1-95% by weight of the total weight of the composition. The composition may be provided in a dosage form that is suitable for the oral, inhalational, parenteral, intravenous, intramuscular, intraperitoneal, intraarticular, inthrathecal, systemic, nasal, buccal, vaginal, rectal, ophthalmic, or subcutaneous administration route. Thus, the composition may be in the form of, e.g., tablets, capsules, pills, powders, granules, suspensions, emulsions, solutions, gels including hydrogels, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

### Controlled Release Formulations

Administration of a combination of the invention in which one or both of the active agents is formulated for controlled release is useful where the tricyclic compound or the steroid, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to maximize the contribution of each agent, when used together, to an amount of that is therapeutically effective for treating pain. Accordingly, a sustained release formulation may be used to avoid frequent dosing that may be required in order to sustain the plasma levels of both agents at a therapeutic level. For example, in preferable oral pharmaceutical compositions of the invention, half-life and mean residency times from 10 to 20 hours for one or both agents of the combination of the invention are observed.

Many strategies can be pursued to obtain controlled release in which the rate of release outweighs the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes. The release mechanism can be controlled such that the tricyclic compound and/or steroid are released at period intervals, the release could be simultaneous, or a delayed release of one of the agents of the combination can be affected, when the early release of one particular agent is preferred over the other. The release mechanism can also be controlled such that one or both of the compounds are released immediately and after a delay.

The tricyclic compound and corticosteroid can be formulated, for example, to be administered once daily during the nighttime. In this example, the tricyclic compound is formulated for immediate release or controlled release (e.g., released over a period of one to two hours, two to four hours, or four to eight hours), and the corticosteroid is formulated for delayed release (e.g., delayed for 2, 3, 4, 5, 6, 7, 8, 9, 10, 11, 12, or more hours). Alternatively, the combination can be formulated such that the tricyclic compound is formulated for immediate release or controlled release (e.g., released over a period of one to two hours, two to four hours, or four to eight hours), and the corticosteroid is formulated for both delayed release (e.g., delayed for 2, 3, 4, 5, 6, 7, 8, 9, 10, 11, 12, or more hours) and either immediate release or controlled released (e.g., released over a period of one to two hours, two to four hours, or four to eight hours).

Controlled release formulations may include a degradable or nondegradable polymer, hydrogel, organogel, or other physical construct that modifies the bioabsorption, half-life or biodegradation of the agent. The controlled release formulation can be a material that is painted or otherwise applied onto the afflicted site, either internally or externally. In one example, the invention provides a biodegradable bolus or implant that is surgically inserted at or near a site of interest (for example, proximal to an arthritic joint). In another example, the controlled release formulation implant can be inserted into an organ, such as in the lower intestine for the treatment inflammatory bowel disease.

Hydrogels can be used in controlled release formulations for the combinations of the present invention. Such polymers are formed from macromers with a polymerizable, non-degradable, region that is separated by at least one degradable region. For example, the water soluble, non-degradable, region can form the central core of the macromer and have at least two degradable regions which are attached to the core, such that upon degradation, the non-degradable regions (in particular a polymerized gel) are separated, as described in U.S. Pat. No. 5,626,863. Hydrogels can include acrylates, which can be readily polymerized by several initiating systems such as eosin dye, ultraviolet or visible light. Hydrogels can also include polyethylene glycols (PEGs), which are highly hydrophilic and biocompatible. Hydrogels can also include oligoglycolic acid, which is a poly(α-hydroxy acid) that can be readily degraded by hydrolysis of the ester linkage into glycolic acid, a nontoxic metabolite. Other chain extensions can include polylactic acid, polycaprolactone, polyorthoesters, polyanhydrides or polypeptides. The entire network can be gelled into a biodegradable network that can be used to entrap and homogeneously disperse combinations of the invention for delivery at a controlled rate.

Chitosan and mixtures of chitosan with carboxymethylcellulose sodium (CMC-Na) have been used as vehicles for the sustained release of drugs, as described by Inouye et al., Drug Design and Delivery 1: 297-305, 1987. Mixtures of these compounds and agents of the combinations of the invention, when compressed under 200 kg/cm², form a tablet from which the active agent is slowly released upon administration to a subject. The release profile can be changed by varying the ratios of chitosan, CMC-Na, and active agent(s). The tablets can also contain other additives, including lactose, CaHPO₄ dihydrate, sucrose, crystalline cellulose, or croscarmellose sodium. Several examples are given in Table 2.

TABLE 2

| Materials | Tablet components (mg) | | | | | | | | | | | |
|---|---|---|---|---|---|---|---|---|---|---|---|---|
| Active agent | 20 | 20 | 20 | 20 | 20 | 20 | 20 | 20 | 20 | 20 | 20 | 20 |
| Chitosan | 10 | 10 | 10 | 10 | 10 | 20 | 3.3 | 20 | 3.3 | 70 | 40 | 28 |
| Lactose | | 110 | | | | 220 | 36.7 | | | | | |
| CMC-Na | 60 | 60 | 60 | 60 | 60 | 120 | 20 | 120 | 20 | | 30 | 42 |
| CaHPO₄*2H₂O | | | 110 | | | | | 220 | 36.7 | 110 | 110 | 110 |
| Sucrose | 110 | | | | | | | | | | | |
| Crystalline | | | | | 110 | | | | | | | |
| Cellulose | | | | | | | | | | | | |
| Croscarmellose Na | | | | 110 | | | | | | | | |

Baichwal, in U.S. Pat. No. 6,245,356, describes a sustained release oral solid dosage forms that includes agglomerated particles of a therapeutically active medicament (for example, a tricyclic compound/corticosteroid combination or component thereof of the present invention) in amorphous form, a gelling agent, an ionizable gel strength enhancing agent and an inert diluent. The gelling agent can be a mixture of a xanthan gum and a locust bean gum capable of cross-linking with the xanthan gum when the gums are exposed to an environmental fluid. Preferably, the ionizable gel enhancing agent acts to enhance the strength of cross-linking between the xanthan gum and the locust bean gum and thereby prolonging the release of the medicament component of the formulation. In addition to xanthan gum and locust bean gum, acceptable gelling agents that may also be used include those gelling agents well-known in the art. Examples include naturally occurring or modified naturally occurring gums such as alginates, carrageenan, pectin, guar gum, modified starch, hydroxypropylmethylcellulose, methylcellulose, and other cellulosic materials or polymers, such as, for example, sodium carboxymethylcellulose and hydroxypropyl cellulose, and mixtures of the foregoing.

In another formulation useful for the combinations of the invention, Baichwal and Staniforth in U.S. Pat. No. 5,135,757 describe a free-flowing slow release granulation for use as a pharmaceutical excipient that includes from about 20 to about 70 percent or more by weight of a hydrophilic material that includes a heteropolysaccharide (such as, for example, xanthan gum or a derivative thereof) and a polysaccharide material capable of cross-linking the heteropolysaccharide (such as, for example, galactomannans, and most preferably locust bean gum) in the presence of aqueous solutions, and from about 30 to about 80 percent by weight of an inert pharmaceutical filler (such as, for example, lactose, dextrose, sucrose, sorbitol, xylitol, fructose or mixtures thereof). After mixing the excipient with a tricyclic compound/corticosteroid combination, or combination agent, of the invention, the mixture is directly compressed into solid dosage forms such as tablets. The tablets thus formed slowly release the medicament when ingested and exposed to gastric fluids. By varying the amount of excipient relative to the medicament, a slow release profile can be attained.

In another formulation useful for the combinations of the invention, Shell, in U.S. Pat. No. 5,007,790, describe sustained-release oral drug-dosage forms that release a drug in solution at a rate controlled by the solubility of the drug. The dosage form comprises a tablet or capsule that includes a plurality of particles of a dispersion of a limited solubility drug (such as, for example, prednisolone or any other agent of the combination of the present invention) in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles, assuring that drug reaches the stomach in the solution state which is less injurious to the stomach than solid-state drug. The programmed eventual dissolution of the polymer depends upon the nature of the polymer and the degree of crosslinking. The polymer is nonfibrillar and substantially water soluble in its uncrosslinked state, and the degree of crosslinking is sufficient to enable the polymer to remain insoluble for the desired time period, normally at least from about 4 hours to 8 hours up to 12 hours, with the choice depending upon the drug incorporated and the medical treatment involved. Examples of suitable crosslinked polymers that may be used in the invention are gelatin, albumin, sodium alginate, carboxymethyl cellulose, polyvinyl alcohol, and chitin. Depending upon the polymer, crosslinking may be achieved by thermal or radiation treatment or through the use of crosslinking agents such as aldehydes, polyamino acids, metal ions and the like.

Silicone microspheres for pH-controlled gastrointestinal drug delivery that are useful in the formulation of the combinations of the invention have been described by Carelli et al., Int. J. Pharmaceutics 179: 73-83, 1999. The microspheres so described are pH-sensitive semi-interpenetrating polymer hydrogels made of varying proportions of poly(methacrylic acid-co-methylmethacrylate) (Eudragit L100 or Eudragit S100) and crosslinked polyethylene glycol 8000 that are encapsulated into silicone microspheres in the 500 to 1000 μm size range.

Slow-release formulations can include a coating which is not readily water-soluble but which is slowly attacked and removed by water, or through which water can slowly permeate. Thus, for example, the combinations of the invention can be spray-coated with a solution of a binder under continuously fluidizing conditions, such as describe by Kitamori et al., U.S. Pat. No. 4,036,948. Examples of water-soluble binders include pregelatinized starch (e.g., pregelatinized corn starch, pregelatinized white potato starch), pregelatinized modified starch, water-soluble celluloses (e.g. hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum and gelatin, organic solvent-soluble binders, such as cellulose derivatives (e.g., cellulose acetate phthalate, hydroxypropylmethyl-cellulose phthalate, ethylcellulose).

Combinations of the invention, or a component thereof, with sustained release properties can also be formulated by spray drying techniques. In one example, as described by Espositio et al., Pharm. Dev. Technol. 5: 267-78, 2000, prednisolone was encapsulated in methyacrylate microparticles (Eudragit R S) using a Mini Spray Dryer, model 190 (Buchi, Laboratorium Technik AG, Flawil, Germany). Optimal conditions for microparticle formation were found to be a feed (pump) rate of 0.5 mL/min of a solution containing 50 mg prednisolone in 10 mL of acetonitrile, a flow rate of nebulized air of 600 L/hr, dry air temperature heating at 80° C., and a flow rate of aspirated drying air of 28 m³/hr.

Yet another form of sustained release combinations can be prepared by microencapsulation of combination agent particles in membranes which act as microdialysis cells. In such a formulation, gastric fluid permeates the microcapsule walls and swells the microcapsule, allowing the active agent(s) to dialyze out (see, for example, Tsuei et al., U.S. Pat. No. 5,589,194). One commercially available sustained-release system of this kind consists of microcapsules having membranes of acacia gum/gelatine/ethyl alcohol. This product is available from Eurand Limited (France) under the trade name Diffucaps™. Microcapsules so formulated might be carried in a conventional gelatine capsule or tabletted.

A sustained-release formulation useful for corticosteroids is described in U.S. Pat. No. 5,792,476, where the formulation includes 2.5-7 mg of a glucocorticoid as active substance with a regulated sustained-release such that at least 90% by weight of the glucocorticoid is released during a period of about 40-80 min, starting about 1-3 h after the entry of the glucocorticoid into the small intestine of the patient. To make these low dose levels of active substance possible, the active substance, i.e. the glucocorticoid, such as prednisolone or prednisone, is micronised, suitably mixed with known diluents, such as starch and lactose, and granulated with PVP (polyvinylpyrrolidone). Further, the granulate is laminated with a sustained release inner layer resistant to a pH of 6.8 and a sustained release outer layer resistant to a pH of 1.0. The inner layer is made of Eudragit®RL (copolymer of acrylic and methacrylic esters with a low content of quaternary ammonium groups) and the outer layer is made of Eudragit®L (anionic polymer synthesized from methacrylic acid and methacrylic acid methyl ester).

A bilayer tablet can be formulated for a combination of the invention in which different custom granulations are made for each agent of the combination and the two agents are compressed on a bi-layer press to form a single tablet. For example, 100 mg of amoxapine, formulated for a controlled release that results in a amoxapine half-life (t_{1/2}) of 8 to 12 hours and a mean residency time (MRT) of from 10 to 16 hours after administration, may be combined in the same tablet with 3 mg of predinisolone, which is formulated such that the t_{1/2} and MRT approximate those of amoxapine (i.e. 8 to 12 hours and 10 to 16 hours, respectively. In addition to controlling the rate of predsnisolone release in vivo, an enteric or delayed release coat may be included that delays the start of drug release such that the Tₘₐₓ of predsnisolone approximate that of amoxapine.

Cyclodextrins are cyclic polysaccharides containing naturally occurring D(+)-glucopyranose units in an α-(1,4) linkage. Alpha-, beta-, and gamma-cyclodextrins, which contain, respectively, six, seven or eight glucopyranose units, are most commonly used and suitable examples are described in PCT Publication Nos. WO91/11172, WO94/02518, and WO98/55148. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. The side on which the secondary hydroxyl groups are located has a wider diameter than the side on which the primary hydroxyl groups are located. The hydrophobic nature of the cyclodextrin inner cavity allows for the inclusion of a variety of compounds. (Comprehensive Supramolecular Chemistry, Volume 3, J. L. Atwood et al., eds., Pergamon Press (1996); Cserhati, Analytical Biochemistry 225: 328-32, 1995; Husain et al., Applied Spectroscopy 46: 652-8, 1992. Cyclodextrins have been used as a delivery vehicle of various therapeutic compounds by forming inclusion complexes with various drugs that can fit into the hydrophobic cavity of the cyclodextrin or by forming non-covalent association complexes with other biologically active molecules. U.S. Pat. No. 4,727,064 describes pharmaceutical preparations consisting of a drug with substantially low water solubility and an amorphous, water-soluble cyclodextrin-based mixture in which the drug forms an inclusion complex with the cyclodextrins of the mixture.

Formation of a drug-cyclodextrin complex can modify the drug's solubility, dissolution rate, bioavailability, and/or stability properties. For example, cyclodextrins have been described for improving the bioavailability of prednisolone, as described by Uekama et al., J. Pharm Dyn. 6:124-127, 1983. A β-cyclodextrin/prednisolone complex can be prepared by adding both components to water and stirring at 25° C. for 7 days. The resultant precipitate recovered is a 1:2 prednisolone/cyclodextrin complex.

Sulfobutylether-β-cyclodextrin (SBE-β-CD, commercially available from CyDex, Inc, Overland Park, Kans., USA and sold as CAPTISOL®) can also be used as an aid in the preparation of sustained-release formulations of agents of the combinations of the present invention. For example, a sustained-release tablet has been prepared that includes prednisolone and SBE-β-CD compressed in a hydroxypropyl methylcellulose matrix (see Rao et al., J. Pharm. Sci. 90: 807-16, 2001).

Polymeric cyclodextrins have also been prepared, as described in U.S. Patent Application Publication Nos. 2003/0017972 and 2003/0008818. The cyclodextrin polymers so formed can be useful for formulating agents of the combinations of the present invention. These multifunctional polymeric cyclodextrins are commercially available from Insert Therapeutics, Inc., Pasadena, Calif., USA.

As an alternative to direct complexation with agents, cyclodextrins may be used as an auxiliary additive, e.g. as a carrier, diluent or solubiliser. Formulations that include cyclodextrins and other agents of the combinations of the present invention (i.e., tricyclic compounds and/or steroids) can be prepared by methods similar to the preparations of the cyclodextrin formulations described herein.

In one embodiment, the invention features a tricyclic antidepressant and a corticosteroid formulated in separate beads that are included in a single capsule. In this embodiment the prednisolone may be present in delayed release beads have a 2 hour delayed release profile. This formulation contains nonpareil beads of microcrystalline cellulose coated with an aqueous suspension of prednisolone and PVP (Kollidon 30) using a fluid bed processor. The drug-layered beads are further coated using an aqueous suspension of an enteric polymer, Eudragit L30D55 with PEG 6000 and talc.

In another embodiment, the prednisolone is present in controlled release beads have a 6-8 hour sustained release profile. This formulation contains nonpareil beads of microcrystalline cellulose coated with an aqueous suspension of prednisolone and PVP (Kollidon 30) in a fluid bed processor. The drug layered beads are further coated using an aqueous suspension of the controlled release polymer ethyl cellulose (Surelease™) with HPMC and glycerin.

### Delivery of Compounds

It is not intended that administration of compounds be limited to a single formulation and delivery method for all compounds of a combination. The combination can be administered using separate formulations and/or delivery methods for each compound of the combination using, for example, any of the above-described formulations and methods. In one example, a first agent is delivered orally, and a second agent is delivered intravenously.

### Dosages

The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

Continuous daily dosing with the combinations of the invention may not be required. A therapeutic regimen may require cycles, during which time a drug is not administered, or therapy may be provided on an as needed basis during periods of acute pain.

As described above, the compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

For prednisolone adapted for oral administration for systemic use, the daily dosage is normally about 0.05-200 mg (0.7-2800 mcg/kg), preferably about 0.1-60 mg (1-850 mcg/kg), and more preferably about 0.1-5 mg (4-70 mcg/kg). Because of the enhancing effect exhibited by tricyclic compounds on prednisolone anti-pain activity, low dosages of prednisolone (e.g., 0.2, 0.4, 0.6, 0.8, 1, 2, 3, 4, 5, 6, 7, 8, 9, or 10 mg/day), when combined with a tricyclic compound, can be effective in treating pain. Administration one to four times daily is desirable. Prednisolone may be administered for one day to one year, and may even be for the life of the patient. Dosages up to 200 mg per day may be necessary.

### Additional Compounds

In certain embodiments of the invention, the compositions, kits, and methods of the invention may include one or more compounds selected from: anti-convulsants, muscle relaxants, pregabalin, ketamide, analgesics (e.g., opiods, NSAIDs, COX-2 inhibitors), other antidepressants (e.g., SSRIs), cannibinoids, sedatives, and anti-anxiety drugs.

Anti-Convulsants

The anticonvulsants are used in prevention of the occurrence of epileptic seizures. The goal of an anticonvulsant is to suppress the rapid and excessive firing of neurons that start a seizure. Many anticonvulsants block sodium (Na+) channels, calcium (Ca2+) channels, AMPA receptors, or NMDA receptors. Some anticonvulsants inhibit the metabolism of GABA or increase its release.

Anti-convulsants include barbiturates (e.g., amobarbital, aprobarbital, barbital, butabarbital, butalbital, hexobarbital, methohexital, pentobarbital, secobarbital, sodium thiopental, talbutal, thiobarbital, Phenobarbital, methylphenobarbital, metharbital, barbexaclone), benzodiazepines (e.g., alprazolam, bromazepam, chlordiazepoxide, cinolazepam, clonazepam, clorazepate, diazepam, estazolam, flunitrazepam, flurazepam, halazepam, ketazolam, loprazolam, lorazepam, lormetazepam, medazepam, midazolam, nitrazepam, nordazepam, oxazepam, phenazepam, pinazepam, prazepam, quazepam, temazepam, tetrazepam, and triazolam), carboxamide (e.g., carbamazepine and oxcarbazepine), vigabatrin, progabide, and tiagabine topiramate, gabapentin, pregabalin, hydantoins (e.g., ethotoin, phenyloin, mephenyloin, and fosphenyloin), oxazolidinediones (e.g., paramethadione, trimethadione, ethadione), beclamide, primidone, pyrrolidines (e.g., brivaracetam, levetiracetam, and seletracetam), succinimides (e.g., ethosuximide, phensuximide, and mesuximide), sulfonamides (e.g., acetazolamide, sulthiame, methazolamide, and zonisamide), lamotrigine, pheneturide, phenacemide, valpromide, valnoctamide, and valproate.

Muscle Relaxants

A muscle relaxant is a drug that decreases the tone of a muscle. Muscle relaxants include methocarbamol, baclofen, carisoprodol, chlorzoxazone, cyclobenzaprine, dantrolene, metaxalone, orphenadrine, pancuronium, tizanidine, and dicyclomine.

Analgesics

Analgesics are compounds used to treat pain. Analgesics include opiods (e.g., morphine, codeine, thebaine, oxycodone, hydrocodone, dihydrocodeine, hydromorphone, oxymorphone, nicomorphine, methadone, levo-alphacetylmethadol, fentanyl, alfentanil, sufentanil, remifentanil, ketobemidone, carfentanyl, ohmefentanyl, ketobemidone, allylprodine, prodine, PEPAP, propoxyphene, dextropropoxyphene, dextromoramide, bezitramide, piritramide, pentazocine, phenazocine, buprenorphine, butorphanol, nalbufine, levorphanol, levomethorphan, dezocine, etorphine, lefetamine, tilidine, tramadol, naloxone, and naltrexone), NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), acetaminophen, and COX-2 inhibitors (e.g., rofecoxib, celecoxib, valdecoxib, and lumiracoxib).

Selective Serotonin Reuptake Inhibitors

In certain embodiments, a selective serotonin reuptake inhibitor can be used in the compositions, methods, and kits of the invention. By “selective serotonin reuptake inhibitor” or “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Kᵢ(norepinephrine) over Kᵢ(serotonin)) of greater than 100.

SSRIs may be used in connection with the invention include cericlamine (e.g., cericlamine hydrochloride); citalopram (e.g., citalopram hydrobromide); clovoxamine; cyanodothiepin; dapoxetine; escitalopram (escitalopram oxalate); femoxetine (e.g., femoxetine hydrochloride); fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipran (e.g., minlacipran hydrochloride); 6-nitroquipazine; paroxetine (e.g., paroxetine hydrochloride hemihydrate; paroxetine maleate; paroxetine mesylate); sertraline (e.g., sertraline hydrochloride); tametraline hydrochloride; viqualine; and zimeldine (e.g., zimeldine hydrochloride).

Structural analogs of cericlamine are those having the formula:

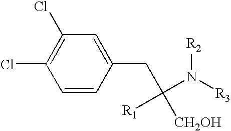

as well as pharmaceutically acceptable salts thereof, wherein R₁ is a C₁-C₄ alkyl and R₂ is H or C₁₋₄ alkyl, R₃ is H, C₁₋₄ alkyl, C₂₋₄ alkenyl, phenylalkyl or cycloalkylalkyl with 3 to 6 cyclic carbon atoms, alkanoyl, phenylalkanoyl or cycloalkylcarbonyl having 3 to 6 cyclic carbon atoms, or R₂ and R₃ form, together with the nitrogen atom to which they are linked, a heterocycle saturated with 5 to 7 chain links which can have, as the second heteroatom not directly connected to the nitrogen atom, an oxygen, a sulphur or a nitrogen, the latter nitrogen heteroatom possibly carrying a C₂₋₄ alkyl.

Exemplary cericlamine structural analogs are 2-methyl-2-amino-3-(3,4-dichlorophenyl)-propanol, 2-pentyl-2-amino-3-(3,4-dichlorophenyl)-propanol, 2-methyl-2-methylamino-3-(3,4-dichlorophenyl)-propanol, 2-methyl-2-dimethylamino-3-(3,4-dichlorophenyl)-propanol, and pharmaceutically acceptable salts of any thereof.

Structural analogs of citalopram are those having the formula:

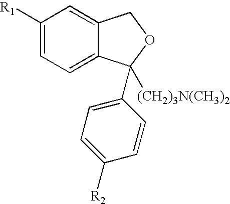

as well as pharmaceutically acceptable salts thereof, wherein each of R₁ and R₂ is independently selected from the group consisting of bromo, chloro, fluoro, trifluoromethyl, cyano and R—CO—, wherein R is C₁₋₄ alkyl.

Exemplary citalopram structural analogs (which are thus SSRI structural analogs according to the invention) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof. Citalopram analogs are also described in U.S. Pat. No. 4,136,193.

Structural analogs of clovoxamine are those having the formula:

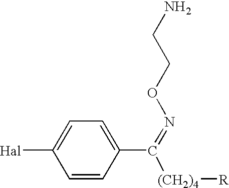

as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Structural analogs of femoxetine are those having the formula:

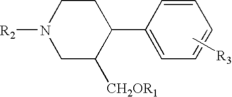

wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Structural analogs of fluoxetine are those compounds having the formula:

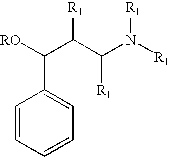

as well as pharmaceutically acceptable salts thereof, wherein each R₁ is independently hydrogen or methyl; R is naphthyl or

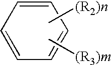

wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate.

Structural analogs of fluvoxamine are those having the formula:

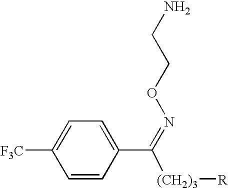

as well as pharmaceutically acceptable salts thereof, wherein R is cyano, cyanomethyl, methoxymethyl, or ethoxymethyl. Analogs of fluvoxamine are also described in U.S. Pat. No. 4,085,225.

Structural analogs of indalpine are those having the formula:

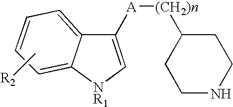

or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3 (piperidyl-4 methyl) ketone; (methoxy-5-indolyl-3) (piperidyl-4 methyl) ketone; (chloro-5-indolyl-3) (piperidyl-4 methyl) ketone; (indolyl-3)-1(piperidyl-4)-3 propanone, indolyl-3 piperidyl-4 ketone; (methyl-1 indolyl-3) (piperidyl-4 methyl) ketone, (benzyl-1 indolyl-3) (piperidyl-4 methyl) ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Structural analogs of indeloxazine are those having the formula:

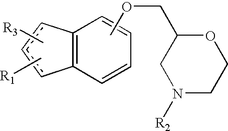

and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Structural analogs of milnacipram are those having the formula:

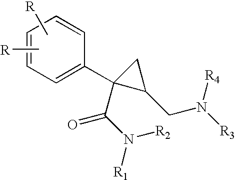

as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Structural analogs of paroxetine are those having the formula:

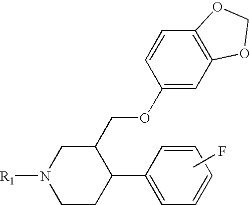

and pharmaceutically acceptable salts thereof, wherein R₁ represents hydrogen or a C₁₋₄ alkyl group, and the fluorine atom may be in any of the available positions.

Structural analogs of sertraline are those having the formula:

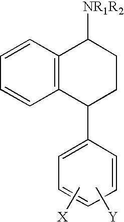

wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers. Sertraline analogs are also described in U.S. Pat. No. 4,536,518.

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

Structural analogs of zimeldine are those compounds having the formula:

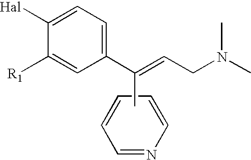

and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof. Zimelidine analogs are also described in U.S. Pat. No. 3,928,369.

Structural analogs of any of the above SSRIs are considered herein to be SSRI analogs and thus may be employed in any of the methods, compositions, and kits of the invention.

Metabolites

Pharmacologically active metabolites of any of the foregoing SSRIs can also be used in the methods, compositions, and kits of the invention. Exemplary metabolites are didesmethylcitalopram, desmethylcitalopram, desmethylsertraline, and norfluoxetine.

Analogs

Functional analogs of SSRIs can also be used in the methods, compositions, and kits of the invention. Exemplary SSRI functional analogs are provided below. One class of SSRI analogs includes SNRIs (selective serotonin norepinephrine reuptake inhibitors), which include venlafaxine, duloxetine, and 4-(2-fluorophenyl)-6-methyl-2-piperazinothieno[2,3-d]pyrimidine.

Structural analogs of venlafaxine are those compounds having the formula:

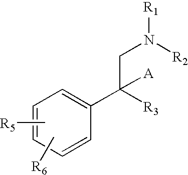

as well as pharmaceutically acceptable salts thereof, wherein A is a moiety of the formula:

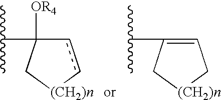

where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Structural analogs of duloxetine are those compounds described by the formula disclosed in U.S. Pat. No. 4,956,388, hereby incorporated by reference. Other SSRI analogs are 4-(2-fluorophenyl)-6-methyl-2-piperazinothieno[2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; 0-desmethylvenlafaxine; WY 45,818; WY 45,881; N-(3-fluoropropyl)paroxetine; Lu 19005; and SNRIs described in PCT Publication No. WO04/004734.

Cannibinoids

Cannabinoids are a group of diterpene C21 compounds present in Cannabis sativa L and include a group of substances that are structurally related to THC or that bind to cannabinoid receptors. Cannibinoids include CP-55940, HU-210, SR141716, SR144528, WIN 55, 212-2, JWH-133, nabilone, levonantradol, marinol, and sativex.

Sedatives

A sedative is a substance that depresses the central nervous system (CNS), resulting in calmness, relaxation, reduction of anxiety, sleepiness, slowed breathing, slurred speech, staggering gait, poor judgment, and slow, uncertain reflexes. Sedatives include chlorpromazine, fluphenazine, haloperidol, loxapine succinate, perphenazine, prochlorperazine, thiothixene, trifluoperazine, clozapine, olanzapine, quetiapine, risperidone, ziprasidone, catnip, Kava Kava, Mandrake, valerian, chloral hydrate, diethyl ether, eszopiclone, ethchlorvynol, ethyl alcohol, gamma-hydroxybutyrate, glutethimide, meprobamate, methaqualone, methyl trichloride, methyprylon, ramelteon, zaleplon, zolpidem, and zopiclone.

### EXAMPLES

### Example 1

### Cytokine Response

A 100 μl suspension of diluted human white blood cells contained within each well of a polystyrene 384-well plate (NalgeNunc) was stimulated to secrete TNFα by treatment with a final concentration of 10 ng/mL phorbol 12-myristate 13-acetate (Sigma, P-1585) and 750 ng/mL ionomycin (Sigma, I-0634). Various concentrations of each test compound were added at the time of stimulation. After 16-18 hours of incubation at 37° C. in a humidified incubator, the plate was centrifuged and the supernatant transferred to a white opaque polystyrene 384-well plate (NalgeNunc, Maxisorb) coated with an anti-TNFα antibody (PharMingen, #551220). After a two-hour incubation, the plate was washed (Tecan PowerWasher 384) with PBS containing 0.1% Tween 20 and incubated for an additional one hour with another anti-TNFα antibody that was biotin labeled (PharMingen, #554511) and HRP coupled to strepavidin (PharMingen, #13047E). After the plate was washed with 0.1% Tween 20/PBS, an HRP-luminescent substrate was added to each well and light intensity measured using a LJL Analyst plate luminometer.

The synergy scores calculated for the various combinations of compounds set forth in Table 3 was calculated by the formula S=log f_{X} log f_{Y}ΣI_{data}(I_{data}−I_{Loewe}), summed over all non-single-agent concentration pairs, and where log f_{X,Y} are the natural logarithm of the dilution factors used for each single agent. This effectively calculates a volume between the measured and Loewe additive response surfaces, weighted towards high inhibition and corrected for varying dilution factors. The synergy score indicates that the combination of the two agents provides greater inhibition of TNFα secretion than would be expected based on the activity of each agent of the combination individually.

TABLE 3
PBMC stimulated with PMA/Ionomycin, synergy score based on TNF-α inhibition

| | Dexa- | Pred- | Bude- | Hydro- | Beta- | Triam- | Di- | Clo- |
|---|---|---|---|---|---|---|---|---|
| Synergy Score | methasone | nisolone | sonide | cortisone | methasone | cinolone | florasone | betasol |
| Amitriptyline | 1.2 | 0.68 | 0.78 | 0.78 | 0.7 | 0.34 | 0.59 | 0.75 |
| Imipramine | 1 | 0.61 | 0.88 | 0.82 | 0.34 | 0.34 | 0.43 | 1 |
| Clomipramine | 1.2 | 0.82 | 0.61 | 0.6 | 0.78 | 0.5 | 0.55 | 0.65 |
| Protriptyline | 1.3 | 1.1 | 1.2 | 0.68 | 0.53 | 0.37 | 0.67 | 0.95 |
| Desipramine | 1.6 | 1.3 | 1 | 0.74 | 0.69 | 0.44 | 0.5 | 0.52 |
| Nortriptyline | 1.7 | 1.1 | 1.6 | 0.72 | 0.83 | 0.88 | | |
| Amoxapine | 1.3 | 1.1 | 0.63 | 0.56 | 0.68 | 0.37 | | |
| Cyproheptadine | 1.3 | 1.3 | 0.56 | 0.75 | 0.8 | 0.57 | | |
| Ketotifen | 1.6 | 0.78 | 0.15 | 0.31 | 0.37 | 0.15 | | |
| Cyclobenzaprine | 1.2 | 0.36 | 0.7 | 0.21 | 0.29 | 0.37 | | |

### Example 2

### Animal Experiments

In the experiments set forth below animals are randomly assigned to experimental groups. All dosing solutions are applied as a single administration. Based on the bioavailability of steroids and tricyclic compounds, one would expect similar results with oral test article administration.

### Carrageenan Pain Model

The carrageenan model is a fast, reliable model used to assess the ability of analgesics to block inflammatory pain. The analgesic effects of test article combinations on pain generation are assayed using the carrageenan-induced pain model in rats. Adult male Sprague-Dawley rats are administered study drugs intraperitoneally (vehicle, tricyclic compound (TCA), steroid or TCA/Steroid test article combinations) once daily for 2 days (day −2 and day −1) and 30 minutes prior to the carrageenan injection on day 0 (time=0). For the carrageenan injection, animals are lightly anesthetized and 0.1 ml of 2% carrageenan is injected into the plantar surface of the right hind paw. The positive control diclofenac at a dose of 25 mg/kg is administered intraperitoneally immediately before the carrageenan injection. Paw volumes (right and left) are measured using a plathysmometer before drug administration on day −2 and serve as a baseline measurement. The paw volumes are measured again two hours post carrageenan injection. The degree of mechanical allodynia is measured by a blinded observer using Von Frey filaments applied to the plantar surface of the hind paws in an increasing numerical order. Each filament increases the force applied on the paw. The filaments are applied until animal paw withdrawal is achieved. This procedure is carried out before drug administration (day −2), on day −1 and on day 0 at time=0, 20, 40, 60, 80 and 120 minutes post-carrageenan. The force (expressed in grams) required for paw withdrawal after carrageenan injection is subtracted from the force required for paw withdrawal before carrageenan injection. Results are expressed as mean change from baseline across five timepoints post carrageenan injection in Table 4.

TABLE 4

| Treatment | 20 minutes | 40 minutes | 60 minutes | 80 minutes | 120 minutes |
|---|---|---|---|---|---|
| Vehicle | 22.50 ± 3.83 | 19.70 ± 6.76 | 25.20 ± 7.06 | 7.90 ± 13.97 | −9.54 ± 18.72 |
| Diclofenac 25 mg/kg | −17.50 ± 11.24 | −74.70 ± 13.27 | −146.70 ± 16.73 | −203.30 ± 15.02 | −259.90 ± 15.14 |
| Nortriptyline 1 mg/kg | 25.50 ± 7.90 | −4.70 ± 8.13 | −59.30 ± 10.6 | −99.70 ± 13.75 | −146.90 ± 19.62 |
| Nortriptyline 3 mg/kg | 7.40 ± 10.10 | −49.20 ± 11.54 | −109.20 ± 11.54 | −169.20 ± 11.54 | −205.40 ± 11.35 |
| Nortriptyline 10 mg/kg | −4.00 ± 8.20 | −68.60 ± 10.46 | −112.80 ± 13.51 | −157.00 ± 17.21 | −185.80 ± 20.18 |
| Nortriptyline 20 mg/kg | −17.60 ± 5.89 | −74.20 ± 7.72 | −130.80 ± 10.37 | −190.80 ± 10.37 | −255.40 ± 14.97 |

### Synergy Calculation

For a synergy calculation, experimental data from the carrageenan study are fitted to a median-effect model and used to calculate the parameters' median-effect dose (Dm), slope (m), correlation coefficient (r), combination index (CI) and dose-reduction index (DRI). In addition, the probability of pain inhibition occurring after single-component (nortriptyline) versus the combination drug treatments are calculated from the negative change from baseline data using a Bayesian sensitivity analysis. The likelihood of pain inhibition is characterized for nortriptyline administered alone (1 mg/kg or 3 mg/kg) versus the fixed-dose combination treatments [prednisolone (0.3 mg/kg):nortriptyline (1 mg/kg) or prednisolone (1 mg/kg):nortriptyline (3 mg/kg)] each evaluated at 60 and 80 minutes for analgesic effect.

Based-on the median-effect model prednisolone/nortriptyline combinations in 1:3 fixed ratios results in significantly lower median-effect doses (Dm) in a rat carrageenan pain model. Prednisolone (Dm) values decrease 10-fold while nortriptyline (Dm) values decrease 4-fold after co-administration compared to the monotherapy. At both dose ratios (0.3:1.0 and 1.0:3.0) strong synergistic interactions (CI<0.75) are predicted by the model. Significant dose reduction indices are calculated for the combination between effect levels ED35 through ED80. Bayesian sensitivity analysis predicts that the 1:3 mg/kg dose ratio has approximately 20% greater likelihood of increasing pain tolerance than does nortriptyline (3 mg) when administered alone. These results are set forth in Table and Table 6 below.

TABLE 5
Carrageenan Model Experiment One: Results expressed as Mean change
from baseline(g) ± SEM

| Treatment | 20 minutes | 40 minutes | 60 minutes | 80 minutes | 120 minutes |
|---|---|---|---|---|---|
| Vehicle | 22.50 ± 3.83 | 19.70 ± 6.76 | 25.20 ± 7.06 | 7.90 ± 13.97 | −9.54 ± 18.72 |
| Diclofenac 25 mg/kg | −17.50 ± 11.24 | −74.70 ± 13.27 | −146.70 ± 16.73 | −203.30 ± 15.02 | −259.90 ± 15.14 |
| Amitriptyline 1 mg/kg | 3.40 ± 3.40 | −49.80 ± 5.19 | −113.80 ± 7.21 | −177.80 ± 10.44 | −231.00 ± 12.33 |
| Amitryptline 3 mg/kg | 11.40 ± 5.82 | −56.60 ± 9.09 | −120.60 ± 11.72 | −173.80 ± 9.90 | −192.10 ± 10.67 |
| Amytriptyline 10 mg/kg | 11.30 ± 5.83 | −41.90 ± 9.86 | −99.10 ± 11.33 | −148.90 ± 12.63 | −177.70 ± 16.01 |
| Amitriptyline 20 mg/kg | 19.90 ± 6.68 | −33.30 ± 7.80 | −93.30 ± 7.80 | −138.60 ± 6.03 | −163..50 ± 5.24 |
| Vehicle | 22.50 ± 3.83 | 19.70 ± 6.76 | 25.20 ± 7.06 | 7.90 ± 13.97 | −9.54 ± 18.72 |
| Diclofenac 25 mg/kg | −17.50 ± 11.24 | −74.70 ± 13.27 | −146.70 ± 16.73 | −203.30 ± 15.02 | −259.90 ± 15.14 |
| Prednisolone 3 mg/kg | 1.10 ± 9.30 | −52.01 ± 12.52 | −101.90 ± 15.02 | −159.70 ± 16.77 | −212.90 ± 20.82 |
| Prednisolone 10 mg/kg | 5.60 ± 6.14 | −44.30 ± 11.47 | −97.50 ± 14.33 | −147.30 ± 16.72 | −193.70 ± 18.07 |
| Prednisolone 20 mg/kg | −29.40 ± 5.10 | −82.60 ± 5.59 | −135.80 ± 7.21 | −189.00 ± 8.41 | −246.20 ± 12.02 |
| Prednisolone 35 mg/kg | −13.60 ± 7.52 | −73.60 ± 7.52 | −130.20 ± 7.26 | −176.60 ± 10.69 | −216.20 ± 11.39 |

TABLE 6
Carrageenan Model Experiment Two: Results expressed as Mean change
from baseline(g) ± SEM

| Treatment | 20 minutes | 40 minutes | 60 minutes | 80 minutes | 120 minutes |
|---|---|---|---|---|---|
| Vehicle | 13..50 ± 11.56 | 27.63 ± 11.32 | 40.25 ± 8.24 | 36.13 ± 7.12 | 51.00 ± 6.39 |
| Diclofenac 25 mg/kg | 5.91 ± 7.99 | 3.89 ± 5.82 | 5.94 ± 10.01 | −2.47 ± 9.56 | −11.26 ± 8.92 |
| Prednisolone 0.3 mg/kg | 4.79 ± 7.92 | 16.77 ± 5.63 | 19.7 ± 5.68 | 24.31 ± 6.40 | 25.57 ± 6.79 |
| Prednisolone 1 mg/kg | 27.60 ± 5.74 | 28.64 ± 5.42 | 25.54 ± 6.04 | 23.77 ± 6.08 | 35.14 ± 5.26 |
| Nortriptyline 1 mg/kg | 16.94 ± 7.35 | 17.01 ± 7.37 | 22.36 ± 4.36 | 29.59 ± 4.80 | 37.64 ± 5.20 |
| Nortriptyline 3 mg/kg | 5.83 ± 6.21 | 5.83 ± 6.21 | 12.87 ± 5.09 | 15.84 ± 5.10 | 35.98 ± 4.09 |
| Amitriptyline 3 mg/kg | −2.20 ± 3.62 | −6.19 ± 5.21 | −1.60 ± 3.83 | 15.70 ± 5.20 | 26.61 ± 4.90 |
| Pred 0.3 mg/kg + Nor 1 mg/kg | 6.94 ± 6.79 | 3.30 ± 7.98 | 13.82 ± 7.48 | 8.88 ± 6.76 | 13.35 ± 7.25 |
| Pred 1 mg/kg + Nor 3 mg/Kg | −2.03 ± 3.67 | 1.12 ± 5.14 | −13.27 ± 8.92 | −17.84 ± 7.07 | 8.86 ± 6.72 |
| Pred 1 mg/kg + Ami 3 mg/Kg | 3.56 ± 3.41 | −3.97 ± 8.52 | −7.83 ± 5.16 | −8.48 ± 5.17 | 8.36 ± 6.71 |
| Vehicle | 4.25 ± 7.70 | 0.00 ± 11.13 | 8.50 ± 10.66 | 9.88 ± 6.55 | 29.50 ± 6.73 |
| Diclofenac 25 mg/kg | −8.50 ± 5.56 | −12.75 ± 6.22 | −8.50 ± 8.50 | −12.75 ± 6.22 | −17.75 ± 6.74 |
| Prednisolone 0.3 mg/kg | 0.00 ± 7.42 | −4.86 ± 4.86 | 0.00 ± 0.00 | 4.86 ± 4.86 | 31.43 ± 5.54 |
| Prednisolone 1 mg/kg | 9.71 ± 6.27 | 0.00 ± 10.49 | 16.14 ± 7.73 | 24.14 ± 7.56 | 12.86 ± 13.38 |
| Nortriptyline 1 mg/kg | 17.00 ± 6.43 | −4.25 ± 4.25 | 32.50 ± 4.96 | 29.75 ± 4.25 | 33.88 ± 5.21 |
| Nortriptyline 3 mg/kg | 17.00 ± 6.43 | 0.00 ± 6.43 | 17.00 ± 6.43 | 0.00 ± 0.00 | 0.00 ± 0.00 |
| Pred 0.3 mg/kg + Nor 1 mg/kg | 9.88 ± 5.43 | −0.00 ± 11.13 | −4.25 ± 10.03 | 14.13 ± 9.50 | 9.88 ± 11.20 |
| Pred 0.3 mg/kg + Nor 3 mg/Kg | 26.88 ± 6.02 | 4.25 ± 7.70 | 0.00 ± 6.43 | 5.63 ± 8.54 | 18.38 ± 9.55 |
| Pred 1 mg/kg + Nor 1 mg/Kg | 0.00 ± 9.09 | 1.38 ± 1.38 | −17.00 ± 9.09 | 0.00 ± 9.09 | 2.75 ± 9.26 |
| Pred 1 mg/kg + Nor 3 mg/Kg | −2.88 ± 10.84 | −0.00 ± 11.13 | −8.50 ± 8.50 | −5.00 ± 10.37 | 18.25 ± 7.05 |

### Durability Calculation

Results of the Carrageenan model demonstrate the enhanced durability of response, represented by the area under the curve (AUC), of the tricyclic compound nortriptlyine in combination with the steroid prednisolone over the individual components. The AUC is determined from 20 minutes through 120 minutes following carrageenan injection and calculated by subtracting the mean baseline force in grams from the force in grams obtained at each time point for each animal (Table 7). The increased AUC demonstrates the improved analagesia benefit over time for the combination when compared to each part of the combination.

TABLE 7

| Treatment | Mean AUC ± SEM |
|---|---|
| Vehicle | 1126 ± 818.09 |
| Diclofenac 25 mg/Kg | 5332 ± 772.96 |
| Prednisolone 0.3 mg/Kg | 2302 ± 634.46 |
| Prednisolone 1 mg/Kg | 1583 ± 574.15 |
| Nortriptyline 1 mg/Kg | 1895 ± 547.01 |
| Nortriptyline 3 mg/Kg | 3082 ± 275.81 |
| Prednisolone 0.3 + Nortriptlyine 1 mg/Kg | 3702 ± 794.78 |
| Prednisolone 1 + Nortriptyline 3 mg/Kg | 5707 ± 493.09 |

In another example, the effects of test article combinations on pain inhibition were assayed using the carrageenan model in rodents. Desipramine was tested alone and in combination at 0.3, 1, and 3 mg/kg and prednisolone was tested similarly at 0.3, 1 and 3 mg/kg IP. Results of the carrageenan model demonstrate enhanced pain inhibition of the tricyclic compound desipramine in combination with the steroid prednisolone over the individual components and vehicle control. The best combination effect was seen with 3 mg/kg prednisolone and 3 mg/kg desipramine.

### Formalin Pain Model

The formalin model is a fast, reliable model used to assess potential analgesics ability to block inflammatory pain. The analgesic effects of test article combinations on pain generation are assayed using the formalin-induced pain model in rats. Adult male Sprague-Dawley rats are administered intraperitoneally study drugs (vehicle, TCA, steroid or TCA/Steroid test article combinations) once daily for 2 days (day −2 and day −1) and 30 minutes prior to the formalin injection day on day 0 (time=0). The positive control morphine at a dose of 20 mg/kg is administered intraperitoneally immediately before the formalin injection. To elicit a pain response, dilute formalin (2% in saline) is injected under the skin of the plantar surface of the right hindpaw. The animal is placed in an observation chamber. The pain response is a characteristic biphasic response pattern of nociceptive behaviors (lifting, licking, and biting of the injected paw). Two distinct periods of nociceptive behaviors/activity are observed and recorded: an early phase (phase I) lasting the first 5-10 min and a late phase (phase II) lasting 20-40 min after the injection of formalin. One hour of data is collected (time spent displaying pain behavior) by a blinded observer using a stopwatch. Results are expressed as the number of pain behaviors per 5 minute periods (Table 8 and Table 9).

TABLE 8
Formalin Model Experiment One: Results Mean number of behaviors per 5
minute block + SEM

| | (Minutes) | | | | | |
|---|---|---|---|---|---|---|
| Treatment | 5 | 10 | 15 | 20 | 25 | 30 |
| Vehicle | 224.0 ± 15.9 | 24.0 ± 15.1 | 57.0 ± 22.2 | 178.0 ± 40.3 | 199.0 ± 40.3 | 246.0 ± 26.7 |
| Morphine | 116.0 ± 39.2 | 19.0 ± 13.7 | 17.0 ± 10.4 | 53.0 ± 32.2 | 77.0 ± 40.2 | 65.0 ± 36.2 |
| 20 mg/kg | | | | | | |
| Amitriptyline | 233.0 ± 10.0 | 52.0 ± 23 | 84.0 ± 28.0 | 49.0 ± 39.0 | 228.0 ± 31.0 | 244.0 ± 19.0 |
| 1 mg/kg | | | | | | |
| Amitriptyline | 206.0 ± 16.2 | 59.0 ± 23.2 | 64.0 ± 21.6 | 166.0 ± 36.7 | 229.0 ± 29.4 | 242.0 ± 27.6 |
| 3 mg/kg | | | | | | |
| Amitriptyline | 217.0 ± 18.1 | 16.0 ± 8.8 | 45.0 ± 24.1 | 84.0 ± 39.0 | 128.0 ± 41.0 | 151.0 ± 40.9 |
| 10 mg/kg | | | | | | |
| Amitriptyline | 168.0 ± 24.3 | 41.0 ± 22.4 | 1.0 ± 0.9 | 13.0 ± 10.2 | 32.0 ± 13.7 | 81.0 ± 34.3 |
| 20 mg/kg | | | | | | |
| | (Minutes) | | | | | |
| Treatment | 35 | 40 | 45 | 50 | 55 | 60 |
| Vehicle | 220.0 ± 32.2 | 202.0 ± 30.2 | 169.0 ± 37.5 | 123.0 ± 33.7 | 65.0 ± 25.8 | 29.0 ± 12.7 |
| Morphine | 66.0 ± 34.4 | 58.0 ± 33.3 | 33.0 ± 22.6 | 11.0 ± 10.3 | 2.0 ± 1.1 | 1.0 ± 0.6 |
| 20 mg/kg | | | | | | |
| Amitriptyline | 240.0 ± 17.0 | 214.0 ± 27.0 | 167.0 ± 31.0 | 63.0 ± 23.0 | 39.0 ± 21.0 | 28.0 ± 23.0 |
| 1 mg/kg | | | | | | |
| Amitriptyline | 210.0 ± 35.9 | 242.0 ± 30.3 | 174.0 ± 36.0 | 119.0 ± 45.0 | 73.0 ± 36.6 | 47.0 ± 26.9 |
| 3 mg/kg | | | | | | |
| Amitriptyline | 152.0 ± 41.1 | 138.0 ± 39.4 | 67.0 ± 26.7 | 70.0 ± 27.8 | 42.0 ± 13.8 | 45.0 ± 25.2 |
| 10 mg/kg | | | | | | |
| Amitriptyline | 93.0 ± 41.3 | 85.0 ± 37.2 | 99.0 ± 34.0 | 82.0 ± 33.8 | 66.0 ± 36.8 | 59.0 ± 31.1 |
| 20 mg/kg | | | | | | |
| | (Minutes) | | | | | |
| Treatment | 5 | 10 | 15 | 20 | 25 | 30 |
| Vehicle | 230.0 ± 19.6 | 47.0 ± 20.5 | 25.0 ± 11.3 | 158.0 ± 38.1 | 196.0 ± 41.3 | 214.0 ± 27.8 |
| Morphine | 120.0 ± 25.2 | 7.0 ± 5.3 | 2.0 ± 1.5 | 2.0 ± 1.1 | 1.0 ± 0.4 | 0.0 ± 0.0 |
| 20 mg/kg | | | | | | |
| Nortriptyline | 185.0 ± 30.0 | 26.0 ± 16.0 | 16.0 ± 16.0 | 33.0 ± 22.0 | 124.0 ± 40.0 | 170.0 ± 46.0 |
| 1 mg/kg | | | | | | |
| Nortriptyline | 248.0 ± 10.6 | 36.0 ± 12.0 | 87.0 ± 32.8 | 159.0 ± 39.7 | 182.0 ± 36.3 | 231.0 ± 32.4 |
| 3 mg/kg | | | | | | |
| Nortriptyline | 227.0 ± 19.3 | 30.0 ± 18.2 | 11.0 ± 6.1 | 39.0 ± 29.4 | 62.0 ± 38.5 | 60.0 ± 34.4 |
| 10 mg/kg | | | | | | |
| Nortriptyline | 158.0 ± 31.6 | 21.0 ± 12.8 | 0.0 ± 0.0 | 9.0 ± 8.8 | 30.0 ± 30.0 | 36.0 ± 29.9 |
| 20 mg/kg | | | | | | |
| | (Minutes) | | | | | |
| Treatment | 35 | 40 | 45 | 50 | 55 | 60 |
| Vehicle | 260.0 ± 28.2 | 268.0 ± 21.3 | 266.0 ± 16.0 | 259.0 ± 18.1 | 217.0 ± 33.8 | 120.0 ± 37.9 |
| Morphine | 0.0 ± 0.4 | 1.0 ± 1.2 | 20.0 ± 20.0 | 29.0 ± 28.6 | 29.0 ± 29.1 | 55.0 ± 36.4 |
| 20 mg/kg | | | | | | |
| Nortriptyline | 181.0 ± 47.0 | 168.0 ± 46.0 | 176.0 ± 45.0 | 191.0 ± 42.0 | 165.0 ± 41.0 | 89.0 ± 34.0 |
| 1 mg/kg | | | | | | |
| Nortriptyline | 253.0 ± 30.7 | 253.0 ± 30.8 | 258.0 ± 28.3 | 212.0 ± 28.3 | 136.0 ± 40.3 | 94.0 ± 41.7 |
| 3 mg/kg | | | | | | |
| Nortriptyline | 99.0 ± 43.0 | 109.0 ± 44.0 | 122.0 ± 43.2 | 96.0 ± 39.1 | 140.0 ± 42.6 | 180.0 ± 40.3 |
| 10 mg/kg | | | | | | |
| Nortriptyline | 33.0 ± 29.2 | 29.0 ± 29.3 | 33.0 ± 26.9 | 46.0 ± 28.2 | 47.0 ± 29.4 | 68.0 ± 37.6 |
| 20 mg/kg | | | | | | |
| | (Minutes) | | | | | |
| Treatment | 5 | 10 | 15 | 20 | 25 | 30 |
| Vehicle | 207.0 ± 17.9 | 38.0 ± 18.6 | 18.0 ± 8.4 | 119.0 ± 34.0 | 165.0 ± 43.9 | 189.0 ± 41.8 |
| Morphine | 125.0 ± 29.6 | 22.0 ± 12.4 | 0.0 ± 0.2 | 16.0 ± 15.9 | 26.0 ± 25.9 | 25.0 ± 24.6 |
| 20 mg/kg | | | | | | |
| Prednisolone | 147.0 ± 22.3 | 11.0 ± 4.4 | 12.0 ± 9.2 | 60.0 ± 31.3 | 85.0 ± 31.2 | 133.0 ± 32.6 |
| 3 mg/kg | | | | | | |
| Prednisolone | 191.0 ± 15.4 | 21.0 ± 13.2 | 69.0 ± 25.3 | 149.0 ± 30.7 | 233.0 ± 22.3 | 257.0 ± 23.2 |
| 10 mg/kg | | | | | | |
| Prednisolone | 216.0 ± 24.1 | 26.0 ± 14.4 | 26.0 ± 15.0 | 62.0 ± 27.6 | 106.0 ± 40.3 | 168.0 ± 38.3 |
| 20 mg/kg | | | | | | |
| Prednisolone | 226.0 ± 17.1 | 19.0 ± 11.3 | 31.0 ± 28.7 | 48.0 ± 27.4 | 119.0 ± 33.7 | 160.0 ± 39.8 |
| 35 mg/kg | | | | | | |
| | (Minutes) | | | | | |
| Treatment | 35 | 40 | 45 | 50 | 55 | 60 |
| Vehicle | 215.0 ± 33.5 | 233.0 ± 24.4 | 204.0 ± 34.3 | 169.0 ± 29.3 | 100.0 ± 38.7 | 85.0 ± 37.7 |
| Morphine | 28.0 ± 25.5 | 1.0 ± 0.7 | 12.0 ± 11.6 | 5.0 ± 4.7 | 1.0 ± 0.7 | 0.0 ± 0.1 |
| 20 mg/kg | | | | | | |
| Prednisolone | 141.0 ± 34.0 | 100.0 ± 33.7 | 108.0 ± 35.7 | 44.0 ± 24.0 | 19.0 ± 16.7 | 14.0 ± 10.2 |
| 3 mg/kg | | | | | | |
| Prednisolone | 218.0 ± 25.9 | 177.0 ± 39.0 | 122.0 ± 41.0 | 104.0 ± 37.0 | 86.0 ± 36.6 | 26.0 ± 11.6 |
| 10 mg/kg | | | | | | |
| Prednisolone | 172.0 ± 33.4 | 177.0 ± 34.8 | 125.0 ± 30.6 | 90.0 ± 35.5 | 34.0 ± 27.8 | 9.0 ± 4.9 |
| 20 mg/kg | | | | | | |
| Prednisolone | 188.0 ± 34.2 | 191.0 ± 38.6 | 170.0 ± 35.2 | 118.0 ± 38.8 | 65.0 ± 32.7 | 27.0 ± 19.7 |
| 35 mg/kg | | | | | | |

TABLE 9
Formalin Model Experiment Two: Results Mean number of behaviors per 5
minute block + SEM

| | (Minutes) | | | | | |
|---|---|---|---|---|---|---|
| Treatment | 5 | 10 | 15 | 20 | 25 | 30 |
| Vehicle | 232.0 ± 20.3 | 16.0 ± 8.4 | 24.0 ± 10.9 | 99.0 ± 32.4 | 205.0 ± 46.3 | 235.0 ± 38.5 |
| Morphine | 64.0 ± 35.3 | 3.0 ± 3.0 | 0.0 ± 0.0 | 0.0 ± 0.4 | 0.0 ± 0.3 | 1.0 ± 1.1 |
| 20 mg/kg | | | | | | |
| Nortriptyline 3 mg/kg | 228.0 ± 25.1 | 28.0 ± 12.3 | 10.0 ± 6.2 | 96.0 ± 43.8 | 162.0 ± 44.0 | 200.0 ± 45.0 |
| Nortriptyline 1 mg/kg | 232.0 ± 16.5 | 13.0 ± 4.9 | 27.0 ± 16.8 | 113.0 ± 44.2 | 165.0 ± 45.1 | 234.0 ± 26.3 |
| Prednisolone 3 mg/kg | 218.0 ± 14.7 | 15.0 ± 6.7 | 6.0 ± 3.4 | 68.0 ± 19.8 | 208.0 ± 35.5 | 214.0 ± 39.8 |
| Prednisolone 1 mg/kg | 224.0 ± 34.1 | 27.0 ± 11.7 | 18.0 ± 15.2 | 115.0 ± 41.9 | 172.0 ± 51.1 | 178.0 ± 52.0 |
| Nortriptyline 3 + Prednisolone | 225.0 ± 9.5 | 11.0 ± 6.8 | 5.0 ± 3.7 | 12.0 ± 10.5 | 69.0 ± 33.7 | 103.0 ± 48.4 |
| 3 mg/kg | | | | | | |
| Nortriptyline 1 + Prednisolone | 203.0 ± 29.1 | 9.0 ± 4.7 | 36.0 ± 29.5 | 86.0 ± 40.6 | 108.0 ± 41.3 | 114.0 ± 34.2 |
| 1 mg/kg | | | | | | |
| | (Minutes) | | | | | |
| Treatment | 35 | 40 | 45 | 50 | 55 | 60 |
| Vehicle | 219.0 ± 47.1 | 208.0 ± 45.1 | 217.0 ± 47.3 | 217.0 ± 47.3 | 215.0 ± 34.5 | 193.0 ± 44.7 |
| Morphine | 1.0 ± 0.9 | 0.0 ± 0.1 | 8.0 ± 8.2 | 25.0 ± 16.4 | 56.0 ± 35.9 | 51.0 ± 36.9 |
| 20 mg/kg | | | | | | |
| Nortriptyline 3 mg/kg | 217.0 ± 47.8 | 216.0 ± 47.3 | 220.0 ± 48.1 | 224.0 ± 42.0 | 247.0 ± 36.2 | 188.0 ± 36.8 |
| Nortriptyline 1 mg/kg | 270.0 ± 10.6 | 281.0 ± 10.4 | 287.0 ± 5.5 | 266.0 ± 16.6 | 192.0 ± 38.3 | 170.0 ± 46.6 |
| Prednisolone 3 mg/kg | 254.0 ± 29.2 | 269.0 ± 14.2 | 258.0 ± 19.9 | 221.0 ± 36.1 | 198.0 ± 38.1 | 133.0 ± 46.8 |
| Prednisolone 1 mg/kg | 174.0 ± 48.7 | 207.0 ± 43.9 | 197.0 ± 45.3 | 192.0 ± 48.4 | 113.0 ± 43.2 | 92.0 ± 29.7 |
| Nortriptyline 3 + Prednisolone | 172.0 ± 50.9 | 167.0 ± 51.5 | 174.0 ± 51.8 | 165.0 ± 49.8 | 156.0 ± 47.8 | 95.0 ± 34.1 |
| 3 mg/kg | | | | | | |
| Nortriptyline 3 + Prednisolone | 167.0 ± 40.9 | 181.0 ± 45.3 | 156.0 ± 48.6 | 134.0 ± 49.2 | 130.0 ± 47.2 | 52.0 ± 26.4 |
| 1 mg/kg | | | | | | |

### Differential Synergy

Results of the carrageenan and formalin studies demonstrate that the tricyclic compounds amitriptyline and nortriptyline exhibit different potencies in blocking pain. The magnitude of these differences varies across the timepoints measured, by dose and by pain model tested. Given that the within-class properties of tricyclic compounds and steroids can vary (i.e., receptor binding affinities and biologic potency, respectively), one can predict, as demonstrated here for nortriptyline and amitriptyline, that the degree of analgesia of the single agents varies within the respective classes. Further, these observations suggest that the synergistic enhancement between a particular TCA/steroid combination can be optimized for pain inhibition, with certain combinations demonstrating no synergy while others prove to be highly synergistic.

### Acetic Acid Pain Model

The acetic acid is a reliable fast model for noxious/visceral pain usually used for screening potential drug candidates for their analgesic activity. The effects of test article combinations on pain generation are assayed using the acetic acid-induced pain model in mice. Adult male ICR mice are administered study drugs intraperitoneally (vehicle, TCA, steroid or TCA/Steroid test article combinations) once daily for 2 days (day −2 and day −1) and 30 minutes prior to the acetic acid injection on day 0 (time=0). The positive control, Morphine at a dose of 5 mg/kg, is administered intraperitoneally 15 minutes before the acetic acid injection. To elicit a pain response, dilute acetic acid (10 ml/kg of 0.6% solution) is injected intraperitoneally into the abdomen. The animal is placed in an observation chamber, and 5 minutes after acetic acid administration, the number of writhes is counted and recorded over a five minute period by a blinded observer. A writhe is considered as a contraction of the abdominal muscles accompanied by an elongation of the body and extension of the hind limb. Results are expressed as number of writhes. In the acetic acid animal model of noxious/visceral pain, the higher dose combination of the TCA/steroid reduces the number of writhes per 5 minutes to the positive control, morphine 5 mg/kg, counts. The result in the higher dose combination group (prednisolone 5+ nortriptyline 1 mg/kg) also significantly decreases (ANOVA, p<0.05) the number of writhes per 5 minutes when compared to the individual agent prednisolone 5 mg/kg.

TABLE 10
Acetic Acid Writing Experiment One: Results expressed as Mean
number of writhes per 5 minutes + SEM

| | Mean # of writhes per 5 |
|---|---|
| Treatment | minutes ± SEM |
| Vehicle | 18.86 ± 2.09 |
| Morphine 5 mg/kg | 1.93 ± 1.53 |
| Prednisolone 1 mg/kg | 8.00 ± 1.70 |
| Prednisolone 5 mg/kg | 14.43 ± 2.07 |
| Prednisolone 10 mg/kg | 12.29 ± 3.83 |
| Prednisolone 35 mg/kg | 11.29 ± 5.22 |
| Vehicle | 18.86 ± 2.09 |
| Morphine 5 mg/kg | 1.93 ± 1.53 |
| Nortriptyline 1 mg/kg | 11.78 ± 3.44 |
| Nortriptyline 5 mg/kg | 7.79 ± 3.81 |
| Nortriptyline 10 mg/kg | 7.57 ± 2.53 |
| Nortriptyline 15 mg/kg | 2.07 ± 1.42 |
| Vehicle | 18.86 ± 2.09 |
| Morphine 5 mg/kg | 1.93 ± 1.53 |
| Amitriptyline 1 mg/kg | 8.71 ± 2.44 |
| Amitriptyline 3 mg/kg | 11.62 ± 2.94 |
| Amitriptyline 5 mg/kg | 4.50 ± 2.04 |
| Amitriptyline 10 mg/kg | 0.79 ± 0.79 |

### Bone Cancer Pain Model

The effects of test article combinations on pain inhibition are assayed using the bone cancer-induced pain model in rodents. Candidate therapies for treatment of tumor-induced bone pain can be evaluated using this rat model. To elicit a pain response, tumor osteolysis is induced through inoculation on day 0 of adult male Sprague-Dawley rats with 3×10⁴ rat mammary carcinoma cells implanted into the left proximal tibia of each animal. The degree of mechanical allodynia is measured using Von Frey filaments applied to the surface of the hindpaws in an increasing numerical order. Each filament increases the force applied on the paw. The filaments are applied until animal paw withdrawal is achieved. Testing is conducted at baseline (day 0) and on days 5, 7, 10, and 14 post-inoculation by a blinded observer. Results are expressed in grams. Rats are administered intraperitoneally study drugs (vehicle, TCA, steroid or TCA/Steroid test article combinations) once daily, 30 minutes prior to testing on days 3 through day 14 (end of study). The positive control morphine at a dose of 5 mg/kg is administered subcutaneously immediately before testing. At the end of the 14-day experimental period, the animals are sacrificed, and the development of tumor osteolysis is confirmed by ex vivo radiography of the injected tibia.

### Diabetic Neuropathy Pain Model

The effects of test article combinations on pain inhibition are assayed using the diabetic neuropathy-induced pain model in rats. The rat diabetic model is a reliable model used to assess the ability of analgesics to block neuropathic pain. To induce diabetes, animals are injected intravenously with steptozocin and citrate buffer. Glucose levels are monitored weekly over a 4 week period. At the end of the monitoring period, only rats that have a blood glucose of 350 mg/dL and higher (i.e. to confirm that diabetes has been induced) are randomized to treatment groups. Adult male Wistar rats are administered study drugs intraperitoneally (vehicle, TCA, steroid or TCA/Steroid test article combinations) 30 minutes prior to pain testing on day 0 (time=0). The positive control gabapentin is administered intraperitoneally immediately before pain testing. The degree of mechanical allodynia is measured by a blinded observer using Von Frey filaments applied to the plantar surface of the hind paws in an increasing numerical order. Each filament increases the force applied on the paw. The filaments are applied until animal paw withdrawal is achieved. Testing is carried out at baseline (prior to STZ administration) before test articles are administered (week 4) and 30 minutes after test articles are administered. Once diabetes is confirmed, Von Frey testing occurs twice a week for two weeks. The force required for paw withdrawal after onset of diabetic neuropathy is compared to the force required for paw withdrawal at baseline. Results are expressed in grams.

### Chronic Constriction Injury Pain Model

The analgesic effects of test article combinations on pain generation are assayed using the Chronic Constriction Injury-induced neuropathic pain model in rats. Adult male Sprague-Dawley rats receive a unilateral chronic constriction injury by means of a loose ligature (Bennett model). Sham-operated animals undergo the same procedure except that no ligatures are placed around the nerve. Seven days following this surgery, the animals are tested for paw withdrawal threshold to mechanical stimuli applied to the affected paw. Animals meeting the criterion of a 50% decrease in mechanical threshold relative to the contralateral paw are randomly assigned to treatment groups [vehicle, 100 mg/kg gabapentin (positive control), TCA, steroid or TCA/Steroid test article combinations dosed intraperitoneally]. Study drugs are administered daily, 30 minutes prior to testing. Behavioral testing (Von Frey, pin prick and infrared thermal) is preformed at baseline (day 7 post surgery) through study day 19. On each day of testing, behavioral responses are examined over 180 minutes. Results are expressed in grams for the Von Frey test and in seconds for both thermal and pin prick tests.

In one example, the effects of test article combinations on pain inhibition were assayed using the chronic constriction injury model described above. Nortriptyline was tested alone and in combination at 1, 3 and 10 mg/kg and prednisolone was tested similarly at 0.3, 1 and 3 mg/kg IP. Results of the chronic constriction injury model demonstrate enhanced pain inhibition of the tricyclic compound nortriptyline in combination with the steroid prednisolone over the individual components and vehicle control. The best combination effect was seen with 0.3 mg/kg prednisolone and 3 mg/kg nortriptyline.

### OTHER EMBODIMENTS

Various modifications and variations of the described methods and compositions of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method of treating pain in a subject comprising orally administering to said subject a corticosteroid and a tricyclic compound, wherein said corticosteroid and tricyclic compound are orally administered simultaneously, or within 14 days of each other, in amounts that together are sufficient to treat said subject.

2. The method of claim 1, wherein the tricyclic compound is selected from the group consisting of imipramine and nortriptyline.

3. The method of claim 1, wherein the corticosteroid is prednisolone.

4. The method of claim 1, wherein said pain is selected from neuropathic pain, diabetic neuropathic pain, postherpetic neuralgia, cancer related pain, and chronic lower back pain.

5. A method of treating pain in a subject comprising orally administering to said subject a compound comprising active and inactive ingredients, wherein said active ingredients consist of a corticosteroid and a tricyclic compound, wherein said corticosteroid and tricyclic compound are orally administered simultaneously, or within 14 days of each other, in amounts that together are sufficient to treat said subject.

6. The method of claim 5, wherein the tricyclic compound is selected from the group consisting of imipramine and nortriptyline.

7. The method of claim 5, wherein the corticosteroid is prednisolone.

8. The method of claim 5, wherein said pain is selected from neuropathic pain, diabetic neuropathic pain, postherpetic neuralgia, cancer related pain, and chronic lower back pain.

9. A kit comprising a tricyclic compound and instructions for orally administering said tricyclic compound and a corticosteroid simultaneously or within 14 days of each other to a subject having pain.

10. A kit comprising a corticosteroid and instructions for orally administering said corticosteroid and a tricyclic compound simultaneously or within 14 days of each other to a subject having pain.

11. A kit comprising a corticosteroid and a tricyclic compound and instructions for orally administering said corticosteroid and tricyclic compound simultaneously or within 14 days of each other to a subject having pain.

12. A method of treating fibromyalgia in a subject comprising orally administering to said subject a corticosteroid and a tricyclic compound, wherein said corticosteroid and tricyclic compound are orally administered simultaneously, or within 14 days of each other, in amounts that together are sufficient to treat said subject.

13. The method of claim 12, wherein said corticosteroid is prednisolone.

14. The method of claim 12, wherein said tricyclic compound is desipramine.

15. A method of treating fibromyalgia in a subject comprising orally administering to said subject a compound comprising active and inactive ingredients, wherein said active ingredients consist of a corticosteroid and a tricyclic compound, wherein said corticosteroid and tricyclic compound are orally administered simultaneously, or within 14 days of each other, in amounts that together are sufficient to treat said subject.

16. The method of claim 15, wherein said corticosteroid is prednisolone.

17. The method of claim 15, wherein said tricyclic compound is desipramine.

18. A composition comprising a corticosteroid and a tricyclic compound, wherein said tricyclic compound is formulated for immediate or controlled release and said corticosteroid is formulated for delayed release.

19. The composition of claim 18 wherein said tricyclic compound is nortriptyline and said corticosteroid is prednisolone.

20. The composition of claim 18, wherein said corticosteroid is formulated to be released with a delay selected from the group consisting of 1, 2, 3, 4, 5, 6, 7, 8, 9, and 10 hours.
